(19) United States
(12) Patent Application Publication
(10) Pub. No.: US 2025/0340520 A1
(43) Pub. Date: Nov. 6, 2025

(54) CRYSTALLINE FORMS OF SODIUM (5-(4-BROMOPHENYL)-6-(2-((5-BROMOPYRIMIDIN-2-YL)OXY)ETHOXY)PYRIMIDIN-4-YL)(SULFAMOYL)AMIDE

(71) Applicant: IDORSIA PHARMACEUTICALS LTD, Allschwil (CH)

(72) Inventors: Martin BOLLI, Allschwil (CH); Markus VON RAUMER, Allschwil (CH)

(21) Appl. No.: 18/868,772

(22) PCT Filed: May 25, 2023
(86) PCT No.: PCT/EP2023/064065

§ 371 Date: Nov. 24, 2024

(30) Foreign Application Priority Data
May 25, 2022 (WO) PCT/EP2022/064253

(51) Int. Cl. C07D 239/47

(52) U.S. Cl. CPC C07D 239/47

(57) ABSTRACT

The present invention concerns novel crystalline forms of aprocitentan sodium salt, processes for the preparation thereof, pharmaceutical compositions comprising said crystalline forms, pharmaceutical compositions prepared from such crystalline forms, and their use as endothelin receptor antagonists.

### FIELD OF THE INVENTION

The present invention concerns novel crystalline forms of sodium (5-(4-bromophenyl)-6-(2-((5-bromopyrimidin-2-yl)oxy)ethoxy)pyrimidin-4-yl)(sulfamoyl)amide (hereinafter also referred to as “COMPOUND”), processes for the preparation thereof, pharmaceutical compositions comprising said crystalline forms, and their use as endothelin receptor inhibitors/antagonists.

Sodium (5-(4-bromophenyl)-6-(2-((5-bromopyrimidin-2-yl)oxy)ethoxy)pyrimidin-4-yl)(sulfamoyl)amide may be represented by formula I

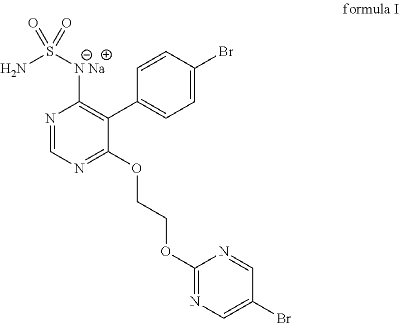

### BACKGROUND

Aprocitentan (({5-(4-bromo-phenyl)-6-[2-(5-bromo-pyrimidin-2-yloxy)-ethoxy]-pyrimidin-4-yl}-sulfamide, COMPOUND in free acid form), also known under the name ACT-132577, is an endothelin receptor inhibitor useful as endothelin receptor antagonist. Aprocitentan is a member of a structural family of compounds previously generically disclosed in WO02/053557. As disclosed in WO2009/024906, aprocitentan is suitable for long-acting pharmaceutical compositions. WO2018/153513 and WO2019/106066 relate to combinations comprising aprocitentan. Certain manufacturing processes relating to aprocitentan are disclosed in WO2015/121397. WO2018/154101 discloses certain crystalline forms of aprocitentan.

Because of its ability to inhibit the endothelin binding, aprocitentan can be used for treatment of endothelin related diseases which are associated with an increase in vasoconstriction, proliferation or inflammation due to endothelin. Examples of such endothelin related diseases are hypertension, pulmonary hypertension, coronary diseases, cardiac insufficiency, renal and myocardial ischemia, renal failure, cerebral ischemia, dementia, migraine, subarachnoidal hemorrhage, Raynaud's syndrome, digital ulcers and portal hypertension. They can also be used in the treatment or prevention of chronic kidney disease (CKD), diabetes, diabetic nephropathy, diabetic retinopathy, diabetic vasculopathy, chronic heart failure and diastolic dysfunction. they can further be used in the treatment or prevention of atherosclerosis, restenosis after balloon or stent angioplasty, inflammation, stomach and duodenal ulcer, cancer, melanoma, prostate cancer, prostatic hypertrophy, erectile dysfunction, hearing loss, amaurosis, chronic bronchitis, asthma, pulmonary fibrosis, gram negative septicemia, shock, sickle cell anemia, glomerulonephritis, renal colic, glaucoma, connective tissue diseases, therapy and prophylaxis of diabetic complications, complications of vascular or cardiac surgery or after organ transplantation, complications of cyclosporin treatment, pain, hyperlipidemia as well as other diseases, presently known to be related to endothelin.

### SUMMARY OF THE INVENTION

It has now been found that certain crystalline forms of COMPOUND (sodium (5-(4-bromophenyl)-6-(2-((5-bromopyrimidin-2-yl)oxy)ethoxy)pyrimidin-4-yl)(sulfamoyl)amide) may be found under certain conditions. Said crystalline forms of COMPOUND are novel and may have advantageous properties in view of the potential use of COMPOUND as active pharmaceutical ingredient. Such advantages may include physical and/or chemical stability; less hygroscopicity; better flow properties; better reproducibility in manufacturing (for example better filtration parameters, better reproducibility of formation, and/or better sedimentation); and/or defined morphology. Such crystalline form of COMPOUND may be particularly suitable in a process of manufacturing of certain pharmaceutical compositions.

### BRIEF DESCRIPTION OF THE FIGURES

FIGS. 1 to 4 show the X-ray powder diffractograms (XRPD) of COMPOUND (sodium (5-(4-bromophenyl)-6-(2-((5-bromopyrimidin-2-yl)oxy)ethoxy)pyrimidin-4-yl)(sulfamoyl)amide) in crystalline forms 1 to 4 as obtained in Examples 1 to 4 (see Table below). The XRPD shows peaks having a relative intensity, as compared to the most intense peak in the diffractogram, of the following percentages (relative peak intensities given in parenthesis) at the indicated angles of refraction 2theta (selected peaks from the range 3-30° 2theta with relative intensity larger than 10% are reported; for Form 3, selected relevant peaks with relative intensity lower than 10% are also reported):

| | | | As obtained |
|---|---|---|---|
| Form | Figure | XRPD peaks | in Example |
| 1 | 1 | 4.3° (53%), 13.1° (21%), 13.5° (26%), | 1 |
| | | 14.8° (15%), 16.6° (12%), 17.2° (64%), | |
| | | 19.0° (12%), 19.5° (19%), 21.1° (53%), | |
| | | 21.6° (71%), 22.6° (100%), 24.0° (24%), | |
| | | 24.6° (25%), 25.0° (30%), 25.8° (17%), | |
| | | and 27.3° (21%) | |
| 2 | 2 | 4.2° (43%), 13.3° (52%), 16.7° (27%), | 2 |
| | | 16.9° (40%), 17.2° (21%), 18.9° (60%), | |
| | | 19.6° (11%), 20.8° (71%), 21.6° (16%), | |
| | | 22.5° (100%), 23.5° (29%), 24.5° (12%), | |
| | | 24.7° (25%), 25.0° (47%), 25.6° (41%), | |
| | | and 27.2° (27%) | |
| 3 | 3 | 3.9° (20%), 11.9° (5%), 15.0° (3%), | 3 |
| | | 15.8° (13%), 17.0° (3%), 19.8° (100%), | |
| | | 22.4° (3%), 23.3° (3%), 23.8° (11%), | |
| | | and 27.8° (19%) | |
| 4 | 4 | 5.9° (59%), 7.7° (26%), 13.5° (25%), | 4 |
| | | 13.9° (45%), 17.0° (26%), 17.6° (50%), | |
| | | 19.4° (27%), 20.0° (24%), 20.7° (99%), | |
| | | 21.9° (100%), 23.5° (36%), and 27.6° | |
| | | (27%) | |

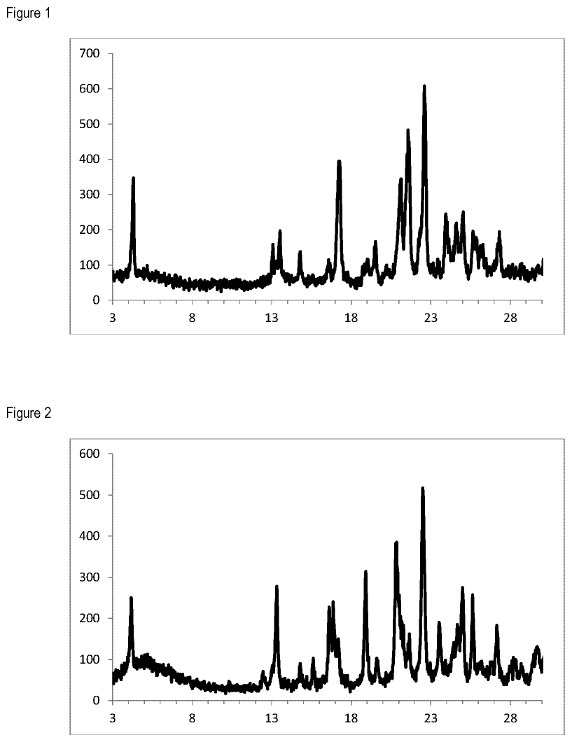

In the X-ray diffractograms of FIG. 1 to 4 the angle of refraction 2theta is plotted on the horizontal axis and the counts on the vertical axis. For avoidance of any doubt, the above-listed peaks describe the experimental results of the diffractograms shown in FIG. 1 to 4. It is understood that, in contrast to the above peak list (see Table above), only a selection of characteristic peaks is required to fully and unambiguously characterize of the COMPOUND in the respective crystalline form of the present invention.

### DETAILED DESCRIPTION

1) A first aspect of the present invention relates to a crystalline form of sodium (5-(4-bromophenyl)-6-(2-((5-bromopyrimidin-2-yl)oxy)ethoxy)pyrimidin-4-yl)(sulfamoyl)amide:

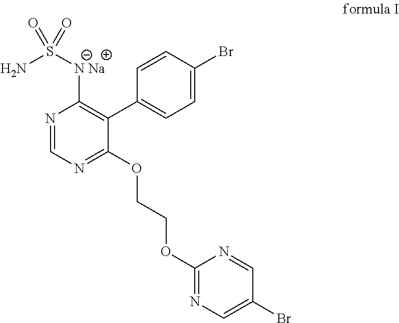

wherein
form 1 of the COMPOUND is characterized by the presence of at least four, or notably at least six, or especially at least eight peaks in the X-ray powder diffractogram at angles of refraction 2θ (2theta) selected from: 4.3°, 13.1°, 13.5°, 14.8°, 17.2°, 19.5°, 21.1°, 21.6°, 22.6°, and 25.0°;
form 2 of the COMPOUND is characterized by the presence of at least four, or notably at least six, or especially at least eight peaks in the X-ray powder diffractogram at angles of refraction 2θ (2theta) selected from: 4.2°, 13.3°, 16.7°, 16.9°, 18.9°, 20.8°, 22.5°, 23.5°, 25.6°, and 27.2°;
form 3 of the COMPOUND is characterized by the presence of at least four, or notably at least six, or especially at least eight peaks in the X-ray powder diffractogram at angles of refraction 2θ (2theta) selected from: 3.9° 11.9°, 15.0°, 15.8°, 17.0°, 19.8°, 22.4°, 23.3°, 23.8°, and 27.8°; or
form 4 of the COMPOUND is characterized by the presence of at least four, or notably at least six, or especially at least eight peaks in the X-ray powder diffractogram at angles of refraction 2θ (2theta) selected from: 5.9°, 7.7°, 13.9°, 17.0°, 17.6°, 19.4°, 20.0°, 20.7°, 21.9°, and 23.5°.

2) One embodiment of the invention relates to a crystalline form of the COMPOUND sodium (5-(4-bromophenyl)-6-(2-((5-bromopyrimidin-2-yl)oxy)ethoxy)pyrimidin-4-yl)(sulfamoyl)amide, wherein
form 1 of the COMPOUND is characterized by the presence of peaks in the X-ray powder diffractogram at the following angles of refraction 2θ (2theta): 4.3°, 17.2°, and 22.6°;
form 2 of the COMPOUND is characterized by the presence of peaks in the X-ray powder diffractogram at the following angles of refraction 2θ (2theta): 13.3°, 20.8°, and 22.5;
form 3 of the COMPOUND is characterized by the presence of peaks in the X-ray powder diffractogram at the following angles of refraction 2θ (2theta): 3.9°, 15.8°, and 19.8°; or
form 4 of the COMPOUND is characterized by the presence of peaks in the X-ray powder diffractogram at the following angles of refraction 2θ (2theta): 5.9°, 20.7°, and 21.9°.

3) Another embodiment of the invention relates to a crystalline form of sodium (5-(4-bromophenyl)-6-(2-((5-bromopyrimidin-2-yl)oxy)ethoxy)pyrimidin-4-yl)(sulfamoyl)amide, wherein
form 1 of the COMPOUND is characterized by the presence of peaks in the X-ray powder diffractogram at the following angles of refraction 2θ (2theta): 4.3°, 17.2°, 21.1°, 21.6°, and 22.6°;
form 2 of the COMPOUND is characterized by the presence of peaks in the X-ray powder diffractogram at the following angles of refraction 2θ (2theta): 4.2°, 13.3°, 18.9°, 20.8°, and 22.5°;
form 3 of the COMPOUND is characterized by the presence of peaks in the X-ray powder diffractogram at the following angles of refraction 2θ (2theta): 3.9°, 15.8°, 19.8°, 23.8°, and 27.8°; or
form 4 of the COMPOUND is characterized by the presence of peaks in the X-ray powder diffractogram at the following angles of refraction 2θ (2theta): 5.9°, 13.9°, 17.6°, 20.7°, and 21.9°.

4) Another embodiment of the invention relates to a crystalline form of sodium (5-(4-bromophenyl)-6-(2-((5-bromopyrimidin-2-yl)oxy)ethoxy)pyrimidin-4-yl)(sulfamoyl)amide, wherein
form 1 of the COMPOUND is characterized by the presence of peaks in the X-ray powder diffractogram at the following angles of refraction 2θ (2theta): 4.3°, 13.1°, 13.5°, 14.8°, 17.2°, 19.5°, 21.1°, 21.6°, 22.60, and 25.00;
form 2 of the COMPOUND is characterized by the presence of peaks in the X-ray powder diffractogram at the following angles of refraction 2θ (2theta): 4.2°, 13.3°, 16.7°, 16.9°, 18.9°, 20.8°, 22.5°, 23.5°, 25.60, and 27.20;
form 3 of the COMPOUND is characterized by the presence of peaks in the X-ray powder diffractogram at the following angles of refraction 2θ (2theta): 3.9°, 11.9°, 15.0°, 15.8°, 17.0°, 19.8°, 22.4°, 23.3°, 23.80, and 27.80; or
form 4 of the COMPOUND is characterized by the presence of peaks in the X-ray powder diffractogram at the following angles of refraction 2θ (2theta): 5.9°, 7.7°, 13.9°, 17.0°, 17.6°, 19.4°, 20.0°, 20.7°, 21.90, and 23.50.

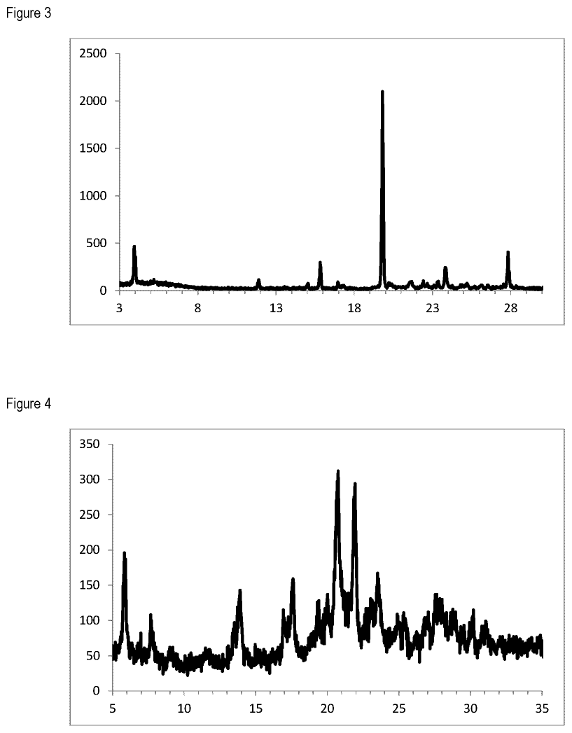

5) Another embodiment of the invention relates to a crystalline form of sodium (5-(4-bromophenyl)-6-(2-((5-bromopyrimidin-2-yl)oxy)ethoxy)pyrimidin-4-yl)(sulfamoyl)amide, which essentially shows the X-ray powder diffractogram of:
form 1 depicted in FIG. 1;
form 2 depicted in FIG. 2;
form 3 depicted in FIG. 3; or
form 4 depicted in FIG. 4.

The term “essentially” as used in embodiment 5) means that at least the major peaks of the diffractogram depicted in said figures, i.e. those having a relative intensity of more than 10%, especially more than 20%, as compared to the most intense peak in the diffractogram, have to be present. However, the person skilled in the art of X-ray powder diffraction will recognize that relative intensities in X-ray powder diffractograms may be subject to strong intensity variations due to preferred orientation effects.

6) A particular embodiment of the invention relates to the a crystalline form of sodium (5-(4-bromophenyl)-6-(2-((5-bromopyrimidin-2-yl)oxy)ethoxy)pyrimidin-4-yl)(sulfamoyl)amide according to any one of embodiments 1) to 5), wherein said crystalline form is form 1.

It is understood that the crystalline forms according to the present invention may comprise non-coordinated and/or coordinated solvent. Coordinated solvent is used herein as term for a crystalline solvate. For the avoidance of doubt, in this application the term “(crystalline) solvate” encompasses non-stoichiometric solvates. Likewise, non-coordinated solvent is used herein as term for physiosorbed or physically entrapped solvent (definitions according to Polymorphism in the Pharmaceutical Industry (Ed. R. Hilfiker, V C H, 2006), Chapter 8: U. J. Griesser: The Importance of Solvates). It is further understood that the crystalline forms of the present invention may contain different amounts of coordinated water as a function of relative humidity and that the X-ray powder diffraction diagram may thus vary with relative humidity. For the avoidance of doubt, the present invention encompasses all crystalline sub-forms of the crystalline form that are reversibly converted into one another depending on relative humidity.

COMPOUND in crystalline form 1 and form 2 may be a hydrate, notably a non-stoichiometric hydrate, especially it may comprise up to about 1.6% (relative to the dry weight of COMPOUND) of coordinated water at 25° C. and 90% relative humidity as determined according to the GVS method described hereinbelow. Form 3 may be a solvate; notably non-stoichiometric solvate; especially comprising solvents such as water and/or organic solvents e.g. methanol. Forms 1, 2 and 3 and may further comprise non-coordinated solvent/s such as water and/or organic solvents e.g. ethyl acetate, methanol, ethanol, n-propanol, isopropanol, and/or cyclopropanol.

For avoidance of any doubt, whenever an embodiment or claim refers to “peaks in the X-ray powder diffractogram at the following angles of refraction 2θ”, said X-ray powder diffractogram is obtained by using combined Cu Kα1 and Kα2 radiation, without Kα2 stripping; and it should be understood that the accuracy of the 2θ values as provided herein is in the range of +/−0.1-0.2°. Notably, when specifying an angle of refraction 2theta (2θ) for a peak in the invention embodiments and the claims, the 2θ value given is to be understood as an interval from said value minus 0.2° to said value plus 0.2° (2θ+/−0.2°); and especially from said value minus 0.1° to said value plus 0.1° (2θ+/−0.1°).

When defining the presence of a peak in e.g. an X-ray powder diffractogram, a common approach is to do this in terms of diagram depicted (S=signal, N=noise). According to this definition, when stating that a peak has to be present in an X-ray powder diffractogram, it is understood that the peak in the X-ray powder diffractogram is defined by having an S/N ratio (S=signal, N=noise) of greater than x (x being a numerical value greater than 1), usually greater than 2, especially greater than 3.

Especially, said crystalline form of sodium (5-(4-bromophenyl)-6-(2-((5-bromopyrimidin-2-yl)oxy)ethoxy)pyrimidin-4-yl)(sulfamoyl)amide according to any one of embodiments 1) to 5) refers to the COMPOUND in crystalline form 1 or crystalline form 4 (notably crystalline form 1) as defined therein.

7) Another aspect of the present invention relates to crystalline form 1 of COMPOUND, according to any one of embodiments 1 to 6, obtainable by a process comprising
a) reacting {5-(4-bromo-phenyl)-6-[2-(5-bromo-pyrimidin-2-yloxy)-ethoxy]-pyrimidin-4-yl}-sulfamide (i.e. COMPOUND in free acid form) with a (notably about 1 eq.) sodium containing base in (notably about 5 to about 15 vol.; especially about 7 to about 11 vol.; in particular about 9.0 vol.; of) a first solvent selected from C₁₋₆-alkyl-OH;
b) optionally distilling off part of the first solvent (notably until a total volume of about 1 to about 7 vol.; especially about 2 to about 6 vol.; in particular about 4 vol.);
c) adding (notably from about 5 to about 15 vol.; especially about 7 to about 12 vol.; in particular about 10.0 vol.; of) a second solvent selected from C₁₋₄-alkyl acetate;
d) optionally distilling off part of the mixture of the first and the second solvent (notably until a total volume of about 1 to about 11 vol.; especially about 3 to about 9 vol.; in particular about 6 vol.); and
e) isolating crystalline form 1.

8) Another aspect of the present invention relates to a process for manufacture of the COMPOUND in crystalline form 1 according to any one of embodiments 1 to 5, said process comprising the steps as defined in embodiment 7).

The term “sodium containing base” as used herein refers to an organic and/or inorganic Brønsted base comprising sodium; notably, wherein said base is capable of accepting protons (H⁺ ions) from COMPOUND in its free acid form such that COMPOUND is obtained. Suitable Brønsted bases may be sodium alkoxide/s (such as sodium methoxide, sodium ethoxide, sodium n-propoxide, sodium iso-propoxide, or sodium butoxide), sodium hydride, sodium oxide, and/or sodium hydroxide; notably sodium alkoxide/s, sodium hydride, or sodium hydroxide; in particular sodium methoxide.

The term “alkyl”, as used herein alone or in combination, refers to a saturated, straight, or branched, acyclic or cyclic (notably acyclic) hydrocarbon containing one to six carbon atoms. The term “C_{x-y}-alkyl” (x and y each being an integer), as used herein alone or in combination, refers to an alkyl as defined hereinabove comprising x to y carbon atoms. Thus, the term “C₁₋₆-alkyl”, as used herein alone or in combination, refers to C_{x-y}-alkyl as defined hereinabove comprising one to six carbon atoms. Examples of C₁₋₆-alkyl groups are methyl, ethyl, n-propyl, iso-propyl, n-butyl, tert-butyl, sec-butyl, iso-butyl, n-pentyl, n-hexyl, cyclopropyl, cyclopentyl, or cyclohexyl. Examples of C₁₋₆-alkyl-OH [i.e. alkanol comprising 1 to 6 (notably 1 to 4; especially 1 or 2) carbon atoms] as used herein are methanol, ethanol, n-propanol, isopropanol, n-butanol, tert-butanol, cyclopropanol, cyclobutanol, or a mixture thereof; notably methanol, ethanol, n-propanol, isopropanol, cyclopropanol, or a mixture thereof; especially such C₁₋₆-alkyl-OH refers to methanol.

The term “sodium alkoxide” as used herein refers to a compound of the formula C₁₋₆-alkyl-ONa.

The term “C₁₋₄-alkyl acetate” as used herein refers to a compound of the formula C₁₋₄-alkyl-O—C(═O)CH₃, wherein C₁₋₆-alkyl refers to C_{x-y}-alkyl as defined hereinabove comprising one to six carbon atoms. Examples are methyl acetate, ethyl acetate, propyl acetate, isopropyl acetate, butyl acetate, tert-butyl acetate, or a mixture thereof; especially ethyl acetate.

It is understood that any one of steps b) or d) independently may be performed under normal or reduced pressure; notably under reduced pressure (e.g. at 100 mbar).

It is further understood that isolating the crystalline form 1 of step e) of any one of embodiments 7) or 8) refers to the process of separating the solid phase of a solid-liquid mixture from its liquid phase. It is understood that said isolation may be performed by any method for solid-liquid separation such as filtration (e.g. gravity filtration or vacuum filtration). It is further understood that the process of isolation may further comprise washing (notably with ethyl acetate) and/or drying (notably at a temperature of up to about 70° C.).

In a preferred variant of any one of embodiments 7) or 8) step b) is not optional. In another preferred variant of any one of embodiments 7) or 8) step d) is not optional. Yet in another preferred variant of any one of embodiments 7) or 8) step b) and step d) are not optional.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C., notably to an interval extending from Y minus 3° C. to Y plus 3° C. Room temperature means a temperature of about 25° C.

When in the current application the term n equivalent(s) is used wherein n is a number, it is meant and within the scope of the current application that n is referring to about the number n, preferably n is referring to the exact number n.

Whenever the term “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

9) Another embodiment thus relates to a crystalline form, especially the essentially pure crystalline form, of sodium (5-(4-bromophenyl)-6-(2-((5-bromopyrimidin-2-yl)oxy)ethoxy)pyrimidin-4-yl)(sulfamoyl)amide according to any one of embodiments 1) to 7) for use as a medicament (e.g. in the form of pharmaceutical compositions for enteral or parenteral administration).

The crystalline form, especially the essentially pure crystalline form (in particular crystalline form 1), of sodium (5-(4-bromophenyl)-6-(2-((5-bromopyrimidin-2-yl)oxy)ethoxy)pyrimidin-4-yl)(sulfamoyl)amide according to any one of embodiments 1) to 7) may be used as a medicament as a single component, as a mixture with other crystalline forms and/or the amorphous form of COMPOUND.

The term “essentially pure” is understood in the context of the present invention to mean especially that at least 90, preferably at least 95, and most preferably at least 99 percent by weight of COMPOUND is present in the crystalline form of the present invention.

10) Another embodiment relates to the use of a crystalline form of sodium (5-(4-bromophenyl)-6-(2-((5-bromopyrimidin-2-yl)oxy)ethoxy)pyrimidin-4-yl)(sulfamoyl)amide according to any one of embodiments 1) to 7), in the manufacture of a pharmaceutical composition, wherein said pharmaceutical composition comprises as active ingredient the compound {5-(4-bromo-phenyl)-6-[2-(5-bromo-pyrimidin-2-yloxy)-ethoxy]-pyrimidin-4-yl}-sulfamide, and at least one therapeutically inert excipient.

11) Another embodiment relates to a pharmaceutical composition comprising as active ingredient the crystalline form of sodium (5-(4-bromophenyl)-6-(2-((5-bromopyrimidin-2-yl)oxy)ethoxy)pyrimidin-4-yl)(sulfamoyl)amide, according to any one of embodiments 1) to 7), and at least one therapeutically inert excipient.

12) A further embodiment relates to a pharmaceutical composition according to embodiment 11), wherein said pharmaceutical composition is in form of a tablet or capsule.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the crystalline forms of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants. Examples of pharmaceutical compositions suitable for aprocitentan are disclosed in WO2018/153513 and WO2019/106066.

For avoidance of any doubt, embodiment 10) especially refers to the crystalline form according to any one of embodiments 1) to 7) which is suitable/which is used as final isolation step of COMPOUND (e.g. in order to meet the purity requirements of pharmaceutical production), whereas the final pharmaceutical composition obtained according to embodiment 10) may or may not contain said crystalline form (e.g. because the originally crystalline form of COMPOUND is further transformed during the manufacturing process and/or is dissolved in the pharmaceutically acceptable carrier material(s); thus, in the final pharmaceutical composition, COMPOUND may be present in non-crystalline form, in another crystalline form, in dissolved form, or the like).

13) A further embodiment relates to a method for the preparation of a pharmaceutical composition comprising as active ingredient the compound {5-(4-bromo-phenyl)-6-[2-(5-bromo-pyrimidin-2-yloxy)-ethoxy]-pyrimidin-4-yl}-sulfamide, wherein method comprising admixing sodium (5-(4-bromophenyl)-6-(2-((5-bromopyrimidin-2-yl)oxy)ethoxy)pyrimidin-4-yl)(sulfamoyl)amide according to any one of embodiments 1) to 7), and at least one therapeutically inert excipient; wherein said pharmaceutical composition especially is in solid or liquid form.

The crystalline form of sodium (5-(4-bromophenyl)-6-(2-((5-bromopyrimidin-2-yl)oxy)ethoxy)pyrimidin-4-yl)(sulfamoyl)amide, according to any one of embodiments 1) to 7), and such pharmaceutical compositions according to any of embodiments 11) or 12) are especially useful for the treatment of endothelin related diseases and disorders, notably for the treatment of hypertension, pulmonary hypertension, coronary diseases, cardiac insufficiency, renal and myocardial ischemia, renal failure, cerebral ischemia, dementia, migraine, subarachnoidal hemorrhage, Raynaud's syndrome, digital ulcers or portal hypertension as well as for the treatment or prevention of atherosclerosis, restenosis after balloon or stent angioplasty, inflammation, stomach and duodenal ulcer, cancer, melanoma, prostate cancer, prostatic hypertrophy, erectile dysfunction, hearing loss, amaurosis, chronic bronchitis, asthma, pulmonary fibrosis, gram negative septicemia, shock, sickle cell anemia, glomerulonephritis, renal colic, glaucoma, connective tissue diseases, diabetic complications, complications of vascular or cardiac surgery or after organ transplantation, complications of cyclosporin treatment, pain or hyperlipidemia. The pharmaceutical compositions according to any one of embodiments 11) or 12) are also useful for the treatment of Chronic Kidney Disease (CKD), especially CKD of stages 1 to 4 as defined by the Kidney Disease Improving Global Outcomes (KDIGO) Guidelines (and notably CKD of stage 3 or 4), and in particular CKD (notably of these stages) associated with or caused by hypertension including essential hypertension.

Preferably, the crystalline form of sodium (5-(4-bromophenyl)-6-(2-((5-bromopyrimidin-2-yl)oxy)ethoxy)pyrimidin-4-yl)(sulfamoyl)amide, according to any one of embodiments 1) to 7), and such pharmaceutical compositions according to any one of embodiments 12) or 12) are useful in the treatment of a disease selected from the group consisting of hypertension, pulmonary hypertension, diabetic arteriopathy, heart failure, erectile dysfunction, angina pectoris and CKD [especially CKD of stages 1 to 4 as defined by the Kidney Disease Improving Global Outcomes (KDIGO) Guidelines (and notably CKD of stage 3 or 4); and in particular CKD (notably of these stages) associated with or caused by hypertension including essential hypertension, and/or associated with or caused by diabetes].

Such endothelin related diseases and disorders may in particular be defined as including hypertension including especially difficult to treat/resistant hypertension; ischemic heart diseases including angina pectoris, coronary diseases, and myocardial ischemia; cardiac insufficiency; chronic kidney disease (CKD) [especially CKD of stages 1 to 4 as defined by the Kidney Disease Improving Global Outcomes (KDIGO) Guidelines (and notably CKD of stage 3 or 4)], and in particular CKD (notably of these stages) caused by/associated with hypertension, or caused by/associated with diabetes (also referred to as diabetic kidney disease (DKD), wherein especially such diabetes is type 2 diabetes); diabetes, and diabetes related diseases such as diabetic arteriopathy, diabetic nephropathy, diabetic retinopathy, or diabetic vasculopathy; reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension); therapy and prophylaxis of diabetic complications; (acute and chronic) renal failure; glomerulonephritis; connective tissue diseases; atherosclerosis; peripheral arterial obliterant disease including chronic peripheral arteriopathy; digital ulcers; diabetic foot ulcers and/or reducing the risk of lower extremety/limb amputations in patients who have diabetes; heart failure (HF) defined as including especially chronic HF, including in particular systolic HF/HF with reduced ejection fraction (HFrEF) (i.e. ejection fraction<about 40%), and diastolic HF/HF with preserved ejection fraction (HFpEF) (i.e. ejection fraction>about 50%); reducing the risk of developing a major cardiovascular event (such as HF, myocardial infarction, stroke, or death from cardiovascular causes) in patients who are at cardiovascular risk (such as patients who have coronary artery disease and/or patients who have demonstrated clinical signs of congestive HF); and diastolic dysfunction.

For avoidance of doubt, the term CKD caused by/associated with diabetes (diabetic kidney disease, DKD) may also include such DKD associated, in addition, with hypertension; wherein especially the diabetes is type 2 diabetes.

Moreover, pharmaceutical compositions according to any one of embodiments 11) or 12) are useful in the treatment of a disease selected from the group consisting of essential hypertension, resistant hypertension, pulmonary hypertension and pulmonary arterial hypertension (and notably in the treatment of resistant hypertension).

Essential hypertension (also called primary hypertension or idiopathic hypertension) is the form of hypertension that by definition has no identifiable cause. It represents a significant global public health concern, contributing to vascular and renal morbidity and to cardiovascular mortality. The diagnosis of essential hypertension is made when the average of multiple systolic blood pressure measurements on 2 or more subsequent visits is consistently equal to or above a certain threshold value T_{SBP}. Individuals with high normal blood pressure tend to maintain pressures that are above average for the general population and are at greater risk for development of definite hypertension and cardiovascular events than the general population. The threshold value T_{SBP} above which treatment is recommended is regularly discussed among clinicians (see e.g. Mancia et al, J. Hypertens. (2013), 31, 1281-1357); accordingly, depending on the patient's general condition and age, T_{SBP} could be 140 or 130 mm Hg, or another suitable value.

The term “resistant hypertension” in the present invention is defined as blood pressure that remains above goal in spite of the concurrent use of 3 antihypertensive agents of different classes. One of the 3 agents should be a diuretic and all agents should be prescribed at optimal/maximal dose amounts. As defined, resistant hypertension patients include patients whose blood pressure is controlled with use of more than 3 medications. That is, patients whose blood pressure is controlled but require 4 or more medications to do so should be considered resistant to treatment (see e.g. Mancia et al, J. Hypertens. (2013), 31, 1281-1357).

The term “diuretic” in the present application refers to loop diuretics including furosemide, bumetanide, ethacrynic acid, torsemide; potassium-sparing diuretics including for example amiloride, and notably including aldosterone antagonists (or alternatively named: mineralocorticoid receptor antagonists (MRA)) such as spironolactone, eplerenone, or finerenone; or aldosterone synthase inhibitors (such as baxdrostat); carbonic anhydrase inhibitors including acetazolamide and methazolamide; and in particular to diuretics of the thiazide class (thiazide-like diuretics) such as especially chlorthalidone, hydrochlorothiazide, chlorothiazide, indapamide, or metolazone. Preferred thiazide-like diuretic are chlorthalidone or hydrochlorothiazide. For avoidance of doubt, even though having a diuretic pharmacological effect, SGLT-2 inhibitors are not encompassed in the term “diuretic” as used herein. Furthermore, certain potassium-sparing diuretics may be considered predominantly for their pharmacological action as mineralocorticoid receptor antagonists (MRA)/aldosterone antagonists (e.g. finerenone), or as aldosterone synthase inhibitors (e.g. baxdrostat), rather than for the diuretic action as potassium-sparing diuretic. Such potassium-sparing diuretics are included herein in the definition as diuretics. It is further understood that background therapy may comprise the combination of several diuretics as defined herein. Particular combinations of diuretics are (i) an aldosterone antagonist in combination with a thiazide-like diuretic; (ii) an aldosterone antagonist in combination with a loop diuretic; (iii) an aldosterone synthase inhibitor in combination with a thiazide-like diuretic; (iv) an aldosterone synthase inhibitor in combination with a loop diuretic; and (v) a loop diuretic in combination with a thiazide-like diuretic. “Diuretic” in particular means in the present application a diuretic of the thiazide class (a thiazide-like diuretic) such as especially chlorthalidone, hydrochlorothiazide, chlorothiazide, indapamide, or metolazone. Preferred diuretics are chlorthalidone or hydrochlorothiazide.

The invention, thus, further relates to COMPOUND, especially a crystalline form of COMPOUND sodium (5-(4-bromophenyl)-6-(2-((5-bromopyrimidin-2-yl)oxy)ethoxy)pyrimidin-4-yl)(sulfamoyl)amide according to any one of embodiments 1) to 7), wherein the COMPOUND/said crystalline form of COMPOUND is used as a medicament, especially for the treatment of resistant hypertension; wherein COMPOUND/said crystalline form is used alone or (preferably) in combination (preferably for simultaneous administration, including a fixed dose combination), e.g. with a diuretic, in particular hydrochlorothiazide (HCTZ or HCT) or chlorthalidone; or with an angiotensin 2 receptor blocker (ARB) such as valsartan; or with an angiotension converting enzyme (ACE) inhibitor such as enalapril; or with an SGLT2 inhibitor such as in particular empagliflozin, or dapagliflozin or canagliflozin. In a sub-embodiment, said combination of COMPOUND/said crystalline form according to any one of embodiments 1) to 7) with a diuretic, in particular hydrochlorothiazide (HCTZ or HCT) or chlorthalidone for the treatment of resistant hypertension; may require further combination (preferably for simultaneous administration, including a fixed dose combination) with one or two additional active ingredients that are antihypertensive agents of different classes (especially a CCB and/or an ARB), in particular valsartan. The invention, thus, especially relates to pharmaceutical compositions comprising COMPOUND/the respective crystalline form of COMPOUND as defined in any one of embodiments 11) or 12); comprising as active ingredients, in addition to COMPOUND/the respective crystalline form of COMPOUND, a diuretic, in particular hydrochlorothiazide (HCTZ or HCT), or chlorthalidone; and optionally further comprising one or two active ingredients that are antihypertensive agents of different classes (especially a CCB and/or an ARB); in particular further comprising valsartan; or comprising as active ingredients, in addition to COMPOUND/the respective crystalline form of COMPOUND, an angiotensin 2 receptor blocker (ARB) such as valsartan; or an angiotension converting enzyme (ACE) inhibitor such as enalapril; or comprising as active ingredients, in addition to COMPOUND/the respective crystalline form of COMPOUND, an SGLT2 inhibitor such as in particular empagliflozin, or dapagliflozin or canagliflozin.

The term “SGLT-2 inhibitor” refers to inhibitors of the sodium glucose cotransporter 2 such as especially atigliflozin, bexagliflozin, canagliflozin, dapagliflozin, empagliflozin, ertugliflozin, henagliflozin, ipragliflozin, luseogliflozin, remogliflozin, sotagliflozin, tianagliflozin, or tofogliflozin (especially canagliflozin, or dapagliflozin, or empagliflozin).

14) A further embodiment relates to a crystalline form of COMPOUND according to any one of embodiments 1) to 7), for use in the treatment of hypertension, pulmonary hypertension, coronary diseases, cardiac insufficiency, renal and myocardial ischemia, renal failure, cerebral ischemia, dementia, migraine, subarachnoidal hemorrhage, Raynaud's syndrome, digital ulcers or portal hypertension as well as for the treatment or prevention of atherosclerosis, restenosis after balloon or stent angioplasty, inflammation, stomach and duodenal ulcer, cancer, melanoma, prostate cancer, prostatic hypertrophy, erectile dysfunction, hearing loss, amaurosis, chronic bronchitis, asthma, pulmonary fibrosis, gram negative septicemia, shock, sickle cell anemia, glomerulonephritis, renal colic, glaucoma, connective tissue diseases, diabetic complications, complications of vascular or cardiac surgery or after organ transplantation, complications of cyclosporin treatment, pain, hyperlipidemia or CKD [especially CKD of stages 1 to 4 as defined by the Kidney Disease Improving Global Outcomes (KDIGO) Guidelines (and notably CKD of stage 3 or 4), and in particular CKD (notably of these stages) caused by/associated with hypertension including essential hypertension].

15) A further embodiment relates to a crystalline form of COMPOUND according to any one of embodiments 1) to 7), for use in the treatment of a disease selected from the group consisting of hypertension, pulmonary hypertension, diabetic arteriopathy, heart failure, erectile dysfunction, angina pectoris and CKD [especially CKD of stages 1 to 4 as defined by the Kidney Disease Improving Global Outcomes (KDIGO) Guidelines (and notably CKD of stage 3 or 4), and in particular CKD (notably of these stages) caused by/associated with hypertension including essential hypertension].

16) A further embodiment relates to a crystalline form of COMPOUND according to any one of embodiments 1) to 7), for use in the treatment of a disease selected from the group consisting of essential hypertension, resistant hypertension, pulmonary hypertension and pulmonary arterial hypertension (and notably for use in the treatment of resistant hypertension).

For avoidance of any doubt, if the COMPOUND especially the crystalline form of COMPOUND according to any one of embodiments 1) to 7), is described as useful for the prevention/prophylaxis or treatment of certain diseases, such crystalline form is likewise suitable for use in the preparation of a medicament for the prevention/prophylaxis or treatment of said diseases. Likewise, such crystalline form is also suitable in a method for the prevention/prophylaxis or treatment of such diseases, comprising administering to a subject (mammal, especially human) in need thereof, an effective amount thereof.

17) A further embodiment relates to the use of a crystalline form of COMPOUND according to any one of embodiments 1) to 7), for the preparation of a medicament intended for the treatment of any one of the diseases or disorders mentioned in embodiment 14).

18) A further embodiment relates to the use of a crystalline form of COMPOUND according to any one of embodiments 1) to 7), for the preparation of a medicament intended for the treatment of any one of the diseases or disorders mentioned in embodiment 15).

19) A further embodiment relates to the use of a crystalline form of COMPOUND according to any one of embodiments 1) to 7), for the preparation of a medicament intended for the treatment of any one of the diseases or disorders mentioned in embodiment 16).

20) A further embodiment relates to a method for the treatment of any one of the diseases or disorders mentioned in embodiment 14), comprising administering to a patient an effective amount of a crystalline form of COMPOUND according to any one of embodiments 1) to 7), or of a pharmaceutical composition according to any one of embodiments 11) or 12).

21) A further embodiment relates to a method for the treatment of any one of the diseases or disorders mentioned in embodiment 15), comprising administering to a patient an effective amount of a crystalline form of COMPOUND according to any one of embodiments 1) to 7), or of a pharmaceutical composition according to any one of embodiments 11) or 12).

22) A further embodiment relates to a method for the treatment of any one of the diseases or disorders mentioned in embodiment 16), comprising administering to a patient an effective amount of a crystalline form of COMPOUND according to any one of embodiments 1) to 7), or of a pharmaceutical composition according to embodiments 11) or 12).

23) Yet another embodiment relates to a crystalline form of COMPOUND according to any one of embodiments 1) to 7), for use in the treatment of a disorder selected from the group consisting of chronic kidney disease (CKD), diabetes, diabetic nephropathy, diabetic retinopathy, diabetic vasculopathy, chronic heart failure and diastolic dysfunction.

24) One sub-embodiment of embodiment 23) relates to a crystalline form of COMPOUND according to any one of embodiments 1) to 7), for use in the treatment of CKD, especially CKD of stages 1 to 4 as defined by the Kidney Disease Improving Global Outcomes (KDIGO) Guidelines (and notably CKD of stage 3 or 4), and in particular CKD (notably of these stages) caused by/associated with hypertension including essential hypertension.

25) Another sub-embodiment of embodiment 23) relates to a crystalline form of COMPOUND according to any one of embodiments 1) to 7), for use in the treatment of diabetes (that is, type 1 or type 2 diabetes).

26) Another sub-embodiment of embodiment 23) relates to a crystalline form of COMPOUND according to any one of embodiments 1) to 7), for use in the treatment of diabetic nephropathy.

27) Another sub-embodiment of embodiment 23) relates to a crystalline form of COMPOUND according to any one of embodiments 1) to 7), for use in the treatment of diabetic retinopathy.

28) Another sub-embodiment of embodiment 23) relates to a crystalline form of COMPOUND according to any one of embodiments 1) to 7), for use in the treatment of diabetic vasculopathy.

29) Another sub-embodiment of embodiment 23) relates to the crystalline form of COMPOUND according to any one of embodiments 1) to 7), for use in the treatment of chronic heart failure.

30) According to one variant of sub-embodiment 29), the chronic heart failure of sub-embodiment 29) is heart failure with preserved ejection fraction.

31) According to another variant of sub-embodiment 29), the chronic heart failure of sub-embodiment 29) is diastolic heart failure.

32) Another sub-embodiment of embodiment 23) relates to a crystalline form of COMPOUND according to any one of embodiments 1) to 7), for use in the treatment of diastolic dysfunction.

33) Preferably, the crystalline form of COMPOUND according to any one of embodiments 23) to 32) will be comprised in a pharmaceutical unit dosage form suitable for the oral administration of 2.5 to 100 mg (in particular 10 to 50 mg, notably 12.5 mg or 25 mg) per day of {5-(4-bromo-phenyl)-6-[2-(5-bromo-pyrimidin-2-yloxy)-ethoxy]-pyrimidin-4-yl}-sulfamide.

34) Preferably, the crystalline form of COMPOUND according to any one of embodiments 23) to 32) will be for use in combination with
an Angiotensin Converting Enzyme (ACE) inhibitor, or an Angiotensin Receptor Blocker (ARB),
and/or a Calcium Channel Blocker (CCB),
or with a pharmaceutically acceptable salt of one of these.

“Angiotensin Converting Enzyme inhibitor” or “ACE inhibitor” in particular means in the present application captopril, enalapril, ramipril, quinapril, perindopril, lisinopril, imidapril or cilazapril, or a pharmaceutically acceptable salt of one of these. A preferred ACE inhibitor is enalapril or a pharmaceutically acceptable salt thereof.

“Angiotensin Receptor Blocker” or “ARB” in particular means in the present application valsartan, losartan, telmisartan, irbesartan, candesartan, olmesartan, azilsartan, or a pharmaceutically acceptable salt of one of these. A preferred ARB is valsartan or a pharmaceutically acceptable salt thereof.

“Calcium Channel Blocker” or “CCB” in particular means in the present application amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, verapamil or diltiazem or a pharmaceutically acceptable salt of one of these. A preferred CCB is amlodipine or a pharmaceutically acceptable salt thereof.

Accordingly, the crystalline form of COMPOUND according to any one of embodiments 23) to 32) can be for use in combination with an ACE inhibitor, or an ARB; and/or a CCB. The corresponding combined treatment may be effected simultaneously, separately, or over a period of time (especially simultaneously).

“Simultaneously”, when referring to an administration type, means in the present application that the administration type concerned consists in the administration of two or more active ingredients and/or treatments at approximately the same time; wherein it is understood that a simultaneous administration will lead to exposure of the subject to the two or more active ingredients and/or treatments at the same time. When administered simultaneously, said two or more active ingredients may be administered in a fixed dose combination, or in an equivalent non-fixed dose combination (e.g. by using two or more different pharmaceutical compositions to be administered by the same route of administration at approximately the same time), or by a non-fixed dose combination using two or more different routes of administration; wherein said administration leads to essentially simultaneous exposure of the subject to the two or more active ingredients and/or treatments. For example, when used in combination with an ACE inhibitor, an ARB, and/or a CCB, the COMPOUND would possibly be used “simultaneously”. Likewise, when used in combination with a diuretic, the COMPOUND would possibly be used “simultaneously”.

“Fixed dose combination”, when referring to an administration type, means in the present application that the administration type concerned consists in the administration of one single pharmaceutical composition comprising the two or more active ingredients.

“Separately”, when referring to an administration type, means in the present application that the administration type concerned consists in the administration of two or more active ingredients and/or treatments at different points in time; wherein it is understood that a separate administration will lead to a treatment phase (e.g. at least 1 hour, notably at least 6 hours, especially at least 12 hours) where the subject is exposed to the two or more active ingredients and/or treatments at the same time; but a separate administration may also lead to a treatment phase where for a certain period of time (e.g. at least 12 hours, especially at least one day) the subject is exposed to only one of the two or more active ingredients and/or treatments. Separate administration especially refers to situations wherein at least one of the active ingredients and/or treatments is given with a periodicity substantially different from daily (such as once or twice daily) administration (e.g. wherein one active ingredient and/or treatment is given e.g. once or twice a day, and another is given e.g. every other day, or once a week or at even longer distances).

By administration “over a period of time” is meant in the present application the subsequent administration of two or more active ingredients and/or treatments at different times. The term in particular refers to an administration method according to which the entire administration of one of the active ingredients and/or treatments is completed before the administration of the other/the others begins. In this way it is possible to administer one of the active ingredients and/or treatments for several months before administering the other active ingredient(s) and/or treatment(s).

35) Also preferably, the crystalline form of COMPOUND according to any one of embodiments 23) to 32), or 33), or when combined with an ACE inhibitor, or an ARB; and/or a CCB according to embodiment 34) will be for use in combination with a diuretic (in particular for use in combination with hydrochlorothiazide (HCT) or chlorthalidone).

Accordingly, the crystalline form of COMPOUND according to any one of embodiments 23) to 32) can be for use in combination with a diuretic (in particular for use in combination with HCT or chlorthalidone). The corresponding combined treatment may be effected simultaneously, separately, or over a period of time (especially simultaneously), as defined hereabove.

36) A further aspect of the invention relates to COMPOUND, sodium (5-(4-bromophenyl)-6-(2-((5-bromopyrimidin-2-yl)oxy)ethoxy)pyrimidin-4-yl)(sulfamoyl)amide, according to any one of embodiments 1) to 7),
for use in the prophylaxis/prevention or treatment of CKD [especially CKD of stages 1 to 4, notably CKD of stage 3 or 4]; and in particular CKD (notably of these stages) caused by/associated with hypertension, and/or caused by/associated with diabetes (DKD); as well as in the prophylaxis/prevention or treatment of acute or chronic renal failure; diabetic nephropathy; or glomerulonephritis;
wherein, in a first sub-embodiment, such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
wherein, in a second sub-embodiment, such use is especially for the treatment of such DKD, including treatment of diabetic nephropathy associated with an elevated serum creatinine and/or proteinuria [especially corresponding to CKD of stages 1 to 4 as defined by the Kidney Disease Improving Global Outcomes (KDIGO) Guidelines (and notably to such CKD of stage 3 or 4)], in patients with type 2 diabetes, especially in such patients presenting in addition a history of hypertension;
for use in the prophylaxis/prevention or treatment of diabetes, and diabetes related diseases such as diabetic arteriopathy, diabetic retinopathy, or diabetic vasculopathy; as well as diabetic complications; for reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension); as well as for use in the prophylaxis/prevention or treatment of diabetic foot ulcers and/or for reducing the risk of lower extremity amputations in patients who have diabetes;
for use in the prophylaxis/prevention or treatment of heart failure (HF) including especially chronic HF, including in particular systolic HF and diastolic HF; for reducing the risk of developing a major cardiovascular event (such as HF, myocardial infarction, stroke, or death from cardiovascular causes) in patients who are at cardiovascular risk (such as patients who have coronary artery disease and/or patients who have demonstrated clinical signs of congestive HF); as well as for use in the prophylaxis/prevention or treatment of ischemic heart diseases including angina pectoris, coronary diseases, and myocardial ischemia; cardiac insufficiency; or diastolic dysfunction;
for use in the treatment of hypertension including especially difficult to treat/resistant hypertension;
for use in the prophylaxis/prevention or treatment of atherosclerosis; as well as of peripheral arterial obliterant disease including chronic peripheral arteriopathy;
for use in the prophylaxis/prevention or treatment of digital ulcers; or
for use in the prophylaxis/prevention or treatment of connective tissue diseases;
   wherein COMPOUND is (intended) to be administered in combination with an SGLT-2 inhibitor, or a pharmaceutically acceptable salt thereof.

In particular, such CKD herein-above may be associated with macroproteinuria (defined as UACR>300 mg/g).

UACR is a biomarker of renal dysfunction, which is monitored in renally impaired patients (Levey et al., Uses of GFR and albuminuria level in acute and chronic kidney disease. N Engl J Med. 2022; 386(22):2120-28].

It is understood that such method of treating CKD (notably CKD of stage 3 or 4) caused by/associated with hypertension and optionally further associated, in addition, with diabetes (diabetic kidney disease (DKD)) likewise refers to treating hypertension including difficult to control and resistant hypertension, wherein said hypertension includes said hypertension causing/associated with such CKD, and wherein said CKD optionally is further associated, in addition to said hypertension, with diabetes.

37) A further embodiment relates to COMPOUND for use according to embodiment 36); wherein COMPOUND is
for use in the prophylaxis/prevention or treatment of CKD [especially CKD of stages 1 to 4, notably CKD of stage 3 or 4], including CKD [especially CKD of stages 1 to 4, notably CKD of stage 3 or 4] caused by/associated with hypertension, and CKD [especially CKD of stages 1 to 4, notably CKD of stage 3 or 4] caused by/associated with diabetes (diabetic kidney disease, DKD);
wherein such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
for use prophylaxis/prevention or treatment of acute renal failure;
for use prophylaxis/prevention or treatment of chronic renal failure;
for use prophylaxis/prevention or treatment of diabetic nephropathy;
for use prophylaxis/prevention or treatment of glomerulonephritis;
for reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension);
for use in the prophylaxis/prevention or treatment of diabetic foot ulcers and/or for reducing the risk of lower extremity amputations in patients who have diabetes;
for use in the prophylaxis/prevention or treatment of heart failure (HF) including especially chronic HF; in particular systolic HF or diastolic HF;
for reducing the risk of developing a major cardiovascular event such as HF, myocardial infarction, stroke, or death from cardiovascular causes in patients who are at cardiovascular risk (such as patients who have coronary artery disease and/or patients who have demonstrated clinical signs of congestive HF);
for use in the prophylaxis/prevention or treatment of diastolic dysfunction;
for use in the treatment of hypertension including especially difficult to treat/resistant hypertension; or
for use in the prophylaxis/prevention or treatment of atherosclerosis; as well as of peripheral arterial obliterant disease including chronic peripheral arteriopathy;
   wherein COMPOUND is (intended) to be administered in combination with an SGLT-2 inhibitor, or a pharmaceutically acceptable salt thereof.

38) A further embodiment relates to COMPOUND for use according to embodiment 36); wherein COMPOUND is
for use in the prophylaxis/prevention or treatment of CKD [especially CKD of stages 1 to 4, notably CKD of stage 3 or 4] caused by/associated with hypertension;
for use in the prophylaxis/prevention or treatment of CKD [especially CKD of stages 1 to 4, notably CKD of stage 3 or 4] caused by/associated with diabetes (DKD);
wherein such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
for use prophylaxis/prevention or treatment of chronic renal failure caused by/associated with hypertension or caused by/associated with diabetes; diabetic nephropathy; or glomerulonephritis caused by/associated with hypertension;
for reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension);
for use in the prophylaxis/prevention or treatment of diabetic foot ulcers and/or for reducing the risk of lower extremity amputations in patients who have diabetes; or
for use in the prophylaxis/prevention or treatment of heart failure (HF) including especially chronic HF; in particular systolic HF or diastolic HF;
   wherein COMPOUND is (intended) to be administered in combination with an SGLT-2 inhibitor, or a pharmaceutically acceptable salt thereof.

39) A further embodiment relates to COMPOUND for use according to embodiment 36); wherein COMPOUND is
for use in the prophylaxis/prevention or treatment of CKD [especially CKD of stages 1 to 4, notably CKD of stage 3 or 4] caused by/associated with hypertension; and/or
for use in the prophylaxis/prevention or treatment of CKD [especially CKD of stages 1 to 4, notably CKD of stage 3 or 4] caused by/associated with diabetes (DKD);
wherein such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension; and/or
for reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension);
   wherein COMPOUND is (intended) to be administered in combination with an SGLT-2 inhibitor, or a pharmaceutically acceptable salt thereof.

40) A further embodiment relates to COMPOUND for use according to embodiment 36); wherein COMPOUND is
for use in the prophylaxis/prevention or treatment of CKD [especially CKD of stages 1 to 4, notably CKD of stage 3 or 4] caused by/associated with hypertension; and/or
for use in the prophylaxis/prevention or treatment of CKD [especially CKD of stages 1 to 4, notably CKD of stage 3 or 4] caused by/associated with diabetes (DKD);
wherein such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
   wherein COMPOUND is (intended) to be administered in combination with an SGLT-2 inhibitor, or a pharmaceutically acceptable salt thereof.

41) A further embodiment relates to COMPOUND for use according to embodiment 36); wherein COMPOUND is
for use in the prophylaxis/prevention or treatment of CKD [especially CKD of stages 1 to 4, notably CKD of stage 3 or 4] caused by/associated with diabetes (DKD);
wherein, in a first sub-embodiment, such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
wherein, in a second sub-embodiment, such use is especially for the treatment of such DKD, including treatment of diabetic nephropathy associated with an elevated serum creatinine and/or proteinuria [especially corresponding to CKD of stages 1 to 4 as defined by the Kidney Disease Improving Global Outcomes (KDIGO) Guidelines (and notably to such CKD of stage 3 or 4)], in patients with type 2 diabetes, especially in such patients presenting in addition a history of hypertension;
   wherein COMPOUND is (intended) to be administered in combination with an SGLT-2 inhibitor, or a pharmaceutically acceptable salt thereof.

42) A further embodiment relates to COMPOUND for use according to embodiment 36); wherein COMPOUND is
for reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension);
   wherein COMPOUND is (intended) to be administered in combination with an SGLT-2 inhibitor, or a pharmaceutically acceptable salt thereof.

43) A further embodiment relates to COMPOUND for use according to any one of embodiments 29) to 35); wherein the SGLT-2 inhibitor, or a pharmaceutically acceptable salt thereof, is notably bexagliflozin, canagliflozin, dapagliflozin, empagliflozin, ertugliflozin, henagliflozin, ipragliflozin, luseogliflozin, sotagliflozin, or tofogliflozin (especially canagliflozin, dapagliflozin, or empagliflozin; in particular empagliflozin), or a pharmaceutically acceptable salt thereof.

It is understood that any embodiment relating to COMPOUND for use in the treatment of certain endothelin related diseases and disorders as specifically defined herein, wherein COMPOUND is (intended) to be administered in combination with another active ingredient (such as one or more diuretics, an ARB or an ACE inhibitor, a CCB, and/or an SGLT-2 inhibitor) also relates
to such active ingredient as disclosed herein (intended) to be administered in combination with COMPOUND for use in the treatment of said endothelin related diseases and disorders;
to the use of COMPOUND for the manufacture of a medicament/a pharmaceutical composition comprising COMPOUND and such active ingredient as disclosed herein, for use in the treatment of said endothelin related diseases and disorders;
to the use of COMPOUND for the manufacture of a medicament/pharmaceutical composition comprising, as active ingredient, COMPOUND for use in the treatment of said endothelin related diseases and disorders; wherein said medicament/pharmaceutical composition is (intended) to be used in combination with such active ingredient as disclosed herein;
to the use of such active ingredient as disclosed herein for the manufacture of a medicament/pharmaceutical composition comprising, as active ingredient, such active ingredient as disclosed herein, for use in the treatment of said endothelin related diseases and disorders; wherein said medicament/pharmaceutical composition is (intended) to be used in combination with COMPOUND;
to the use of a pharmaceutical composition comprising COMPOUND, or a pharmaceutically acceptable salt thereof, and such active ingredient as disclosed herein for the treatment of said endothelin related diseases and disorders;
to a medicament for the prevention or treatment said endothelin related diseases and disorders, said medicament comprising COMPOUND; wherein said medicament is (intended) to be administered in combination with such active ingredient;
to a method of treating said endothelin related diseases and disorders comprising administering to a subject (preferably a human) in need thereof an effective amount of COMPOUND, to be administered in combination with an effective amount of such active ingredient;
to a method of treating said endothelin related diseases and disorders comprising administering to a subject in need thereof an effective amount of a pharmaceutical composition comprising COMPOUND, and such active ingredient as disclosed herein; and
to a method of treating said endothelin related diseases and disorders comprising administering to a subject (preferably a human) in need thereof an effective amount of such active ingredient as disclosed herein to be administered in combination with an effective amount of COMPOUND.

44) The present invention further relates to a crystalline form, especially the essentially pure crystalline form, of sodium (5-(4-bromophenyl)-6-(2-((5-bromopyrimidin-2-yl)oxy)ethoxy)pyrimidin-4-yl)(sulfamoyl)amide according to any one of embodiments 1) to 7), for the prevention or treatment of endothelin related diseases and disorders as defined herein above, wherein said crystalline form of sodium (5-(4-bromophenyl)-6-(2-((5-bromopyrimidin-2-yl)oxy)ethoxy)pyrimidin-4-yl)(sulfamoyl)amide is to be administered in combination with one or more active ingredients [notably at least two (especially three or four) other active ingredients], wherein said one or more active ingredients independently are selected from the following groups of active ingredients:
an angiotenin receptor blocker (especially valsartan, as well as losartan, candesartan, irbesartan, telmisartan, eprosartan, olmesartan, azilsartan, fimasartan), or a pharmaceutically acceptable salt thereof; or
an ACE inhibitor (especially enalapril, ramipril, quinapril, perindopril, lisinopril, benazepril, imidapril, trandolapril, cilazapril), or a pharmaceutically acceptable salt thereof; and/or
a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof; and/or
an aldosterone antagonist such as spironolactone, eplerenone, or finerenone; or an aldosterone synthase inhibitor (such as baxdrostat); and/or
a diuretic selected from:
   loop diuretics including furosemide, bumetanide, ethacrynic acid, or torsemide;
   carbonic anhydrase inhibitors including acetazolamide or methazolamide;
   in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); or
   potassium sparing diuretics which are not aldosterone antagonists or aldosterone synthase inhibitors (an example is amiloride); and/or
a beta blocker (beta-adrenergic blocking agent), especially acebutolol, atenolol, bisoprolol, metoprolol (immediate release or sustained release), nadolol, nebivolol, propranolol (immediate release or sustained release); and/or
an SGLT-2 inhibitor (especially atigliflozin, bexagliflozin, canagliflozin, dapagliflozin, empagliflozin, ertugliflozin, henagliflozin, ipragliflozin, luseogliflozin, remogliflozin, sotagliflozin, or tofogliflozin; or a pharmaceutically acceptable salt thereof; especially bexagliflozin, canagliflozin, dapagliflozin, empagliflozin, ertugliflozin, henagliflozin, ipragliflozin, luseogliflozin, sotagliflozin, or tofogliflozin; in particular canagliflozin, or dapagliflozin, or empagliflozin), or a pharmaceutically acceptable salt thereof.

45) The present invention further relates to a crystalline form, especially the essentially pure crystalline form, of sodium (5-(4-bromophenyl)-6-(2-((5-bromopyrimidin-2-yl)oxy)ethoxy)pyrimidin-4-yl)(sulfamoyl)amide according to any one of embodiments 1) to 7), for the prevention or treatment of endothelin related diseases and disorders as defined herein above, wherein said crystalline form of sodium (5-(4-bromophenyl)-6-(2-((5-bromopyrimidin-2-yl)oxy)ethoxy)pyrimidin-4-yl)(sulfamoyl)amide is to be administered in combination with one or more active ingredients [notably at least two (especially three or four) other active ingredients], wherein said one or more active ingredients independently are selected from the following groups of active ingredients:
an angiotenin receptor blocker (especially valsartan, as well as losartan, candesartan, irbesartan, telmisartan, eprosartan, olmesartan, azilsartan, fimasartan), or a pharmaceutically acceptable salt thereof; or an ACE inhibitor (especially enalapril, ramipril, quinapril, perindopril, lisinopril, benazepril, imidapril, trandolapril, cilazapril), or a pharmaceutically acceptable salt thereof; and/or
a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof; and/or
a loop diuretic including furosemide, bumetanide, ethacrynic acid, torsemide; and/or
a further diuretic selected from
   potassium sparing diuretics which are not aldosterone antagonists or aldosterone synthase inhibitors, such as amiloride;
   aldosterone antagonists such as spironolactone, eplerenone, or finerenone; or aldosterone synthase inhibitors (such as baxdrostat);
   carbonic anhydrase inhibitors including acetazolamide and methazolamide; or
   in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); and/or
a beta blocker (beta-adrenergic blocking agent), especially acebutolol, atenolol, bisoprolol, metoprolol (immediate release or sustained release), nadolol, nebivolol, propranolol (immediate release or sustained release); and/or
an SGLT-2 inhibitor (especially atigliflozin, bexagliflozin, canagliflozin, dapagliflozin, empagliflozin, ertugliflozin, henagliflozin, ipragliflozin, luseogliflozin, remogliflozin, sotagliflozin, or tofogliflozin; or a pharmaceutically acceptable salt thereof; especially bexagliflozin, canagliflozin, dapagliflozin, empagliflozin, ertugliflozin, henagliflozin, ipragliflozin, luseogliflozin, sotagliflozin, or tofogliflozin; in particular canagliflozin, or dapagliflozin, or empagliflozin), or a pharmaceutically acceptable salt thereof.

The term “beta blocker” refers to such beta-adrenergic blocking agent, especially to acebutolol, atenolol, bisoprolol, metoprolol (including immediate release or sustained release formulations), nadolol, nebivolol, and propranolol (including immediate release or sustained release formulations); in particular to atenolol, bisoprolol, metoprolol, nebivolol, and propranolol.

In certain instances, further background treatment may include active ingredients suitable for the treatment of diabetes, such as metformin; and/or insulin; and/or a sulfonylurea (especially glibenclamide), or a pharmaceutically acceptable salt thereof; and/or a DPP-4 inhibitor (especially sitagliptin, vildagliptin, saxagliptin, or linagliptin), or a pharmaceutically acceptable salt thereof; and/or a GLP-1 receptor agonist (especially exenatide, liraglutide, lixisenatide, albiglutide, dulaglutide, taspoglutide, semaglutide, or a dual GLP-1/GIP agonist such as trizepatide); and/or a thiazolidinedione, or a pharmaceutically acceptable salt thereof.

The term “DPP-4 inhibitor” or “DPP-IV inhibitor” refers to inhibitors of dipeptidyl peptidase 4 such as especially sitagliptin, vildagliptin, saxagliptin, and linagliptin, as well as gemigliptin, anagliptin, teneligliptin, alogliptin, trelagliptin, omarigliptin, evogliptin, and dutogliptin.

The term “GLP-1 receptor agonist” refers to agonists of the glucagon-like peptide-1 receptor such as especially exenatide, liraglutide, lixisenatide, albiglutide, dulaglutide, taspoglutide, semaglutide.

The term “dual GLP-1/GIP receptor agonist” refers to dual agonists of the glucagon-like peptide-1 receptor and the glucose-dependent insulinotropic polypeptide (GIP) receptor, such as especially trizepatide.

The term “sulfonylurea” refers especially to glibenclamide (glyburide), glibornuride, gliclazide, glipizide, gliquidone, glisoxepide, glyclopyramide, or glimepiride.

The term “thiazolidinediones” abbreviated as TZD, also known as glitazones, refers to agonists of the PPAR□ (peroxisome proliferator-activated receptor gamma), and refers especially to pioglitazone, rosiglitazone, or lobeglitazone.

Background therapy is preferably to be administered in a dosage corresponding to a tolerated efficacious dose of the respective active ingredient, e.g. when given as a single therapy. In particular, valsartan, or a pharmaceutically acceptable salt thereof, if present, is to be administered in a dosage form suitable for the oral administration of 160 mg or 320 mg per day of valsartan; losartan, or a pharmaceutically acceptable salt thereof, if present, is to be administered in a dosage form suitable for the oral administration of 50 mg or 100 mg per day of losartan; irbesartan, or a pharmaceutically acceptable salt thereof, if present, is to be administered in a dosage form suitable for the oral administration of 75 mg, 150 mg, or 300 mg per day of irbesartan; amlodipine, or a pharmaceutically acceptable salt thereof, if present, is to be administered in a dosage form suitable for the oral administration of 5 mg or 10 mg per day of amlodipine; enalapril, or a pharmaceutically acceptable salt thereof, if present, is to be administered in a dosage form suitable for the oral administration of 2.5 mg to 40 mg per day of enalapril; lisinopril, or a pharmaceutically acceptable salt thereof, if present, is to be administered in a dosage form suitable for the oral administration of 2.5 mg to 40 mg per day of lisinopril; ramipril, or a pharmaceutically acceptable salt thereof, if present, is to be administered in a dosage form suitable for the oral administration of 2.5 mg to 20 mg per day of ramipril; metformin, if present, is to be administered in a dosage form suitable for the oral administration of 500 mg to 2000 mg per day of metformin; glibenclamide if present, is to be administered in a dosage form suitable for the oral administration of 1.25 mg to 5 mg per day of glibenclamide; sitagliptin if present, is to be administered in a dosage form suitable for the oral administration of 25 mg to 100 mg per day of sitagliptin; vildagliptin if present, is to be administered in a dosage form suitable for the oral administration of two times 50 mg per day of vildagliptin; saxagliptin if present, is to be administered in a dosage form suitable for the oral administration of 2.5 mg or 5 mg per day of saxagliptin; linagliptin if present, is to be administered in a dosage form suitable for the oral administration of two times 5 mg per day of linagliptin; bexagliflozin, or a pharmaceutically acceptable salt thereof, if present, is to be administered in a dosage form suitable for the oral administration of 5 to 50 mg (in particular 20 mg) per day of bexagliflozin; canagliflozin, or a pharmaceutically acceptable salt thereof, if present, is to be administered in a dosage form suitable for the oral administration of of 50 to 400 mg (in particular 50 mg, 100 mg, 150 mg, or 300 mg; notably 100 mg, or 300 mg; especially 100 mg) per day of canagliflozin; dapagliflozin, or a pharmaceutically acceptable salt thereof, if present, is to be administered in a dosage form suitable for the oral administration of 1 to 20 mg (in particular 5 mg or 10 mg) per day of dapagliflozin; empagliflozin, or a pharmaceutically acceptable salt thereof, if present, is to be administered in a dosage form suitable for the oral administration of 5 to 50 mg (in particular 10 mg or 25 mg) per day of empagliflozin; ertugliflozin, or a pharmaceutically acceptable salt thereof, if present, is to be administered in a dosage form suitable for the oral administration of 2.5 to 50 mg (in particular 5 mg or 15 mg) per day of ertugliflozin; henagliflozin, or a pharmaceutically acceptable salt thereof, if present, is to be administered in a dosage form suitable for the oral administration of 5 to 100 mg (in particular 25 mg) per day of henagliflozin; ipragliflozin, or a pharmaceutically acceptable salt thereof, if present is to be administered in a dosage form suitable for the oral administration of 10 to 100 mg (in particular 25 mg or 50 mg) per day of ipragliflozin; luseogliflozin, or a pharmaceutically acceptable salt thereof, if present, is to be administered in a dosage form suitable for the oral administration of 1 to 10 mg (in particular 2.5 mg or 5 mg) per day of luseogliflozin; sotagliflozin, or a pharmaceutically acceptable salt thereof, if present, is to be administered in a dosage form suitable for the oral administration of 50 to 500 mg (in particular 75 mg, 200 mg or 400 mg) per day of sotagliflozin, tofogliflozin, or a pharmaceutically acceptable salt thereof, if present, is to be administered in a dosage form suitable for the oral administration of 10 to 50 mg (in particular 20 mg) per day of tofogliflozin.

46) Another embodiment relates to a pharmaceutical composition comprising as active ingredient the crystalline form of the COMPOUND, sodium (5-(4-bromophenyl)-6-(2-((5-bromopyrimidin-2-yl)oxy)ethoxy)pyrimidin-4-yl)(sulfamoyl)amide, according to any one of embodiments 1) to 7), one or more other active ingredient(s) as defined above, e.g. in embodiments 44) or 45), (including especially a diuretic such as an aldosterone antagonist such as finerenone, or an aldosterone synthase inhibitor such as baxdrostat; and/or a thiazide-like diuretic such as HTC or chlorthalidone; and/or an ARB or an ACE inhibitor; and/or a CCB; and/or an SGLT-2 inhibitor) as defined herein above, and at least one therapeutically inert excipient.

47) A further embodiment relates to a pharmaceutical composition according to embodiment 46), wherein said pharmaceutical composition is in form of a tablet or capsule.

48) The invention further relates to a kit comprising
a pharmaceutical composition/a medicament, said pharmaceutical composition/medicament comprising the COMPOUND in crystalline form according to any one of embodiments 1) to 7), and a pharmaceutically acceptable carrier material, and optionally another active ingredient as defined before;
   and instructions how to use said pharmaceutical composition/medicament for the prevention/prophylaxis or the treatment of an endothelin related disease or disorder as defined herein, in combination with (an)other active ingredient(s) (as defined above, e.g. in embodiments 44) or 45), including especially a diuretic such as an aldosterone antagonist such as finerenone, or an aldosterone synthase inhibitor such as baxdrostat; and/or a thiazide-like diuretic such as HTC or chlorthalidone; and/or an ARB or an ACE inhibitor; and/or a CCB; and/or an SGLT-2 inhibitor) as defined herein above.

The amount of the crystalline form of sodium (5-(4-bromophenyl)-6-(2-((5-bromopyrimidin-2-yl)oxy)ethoxy)pyrimidin-4-yl)(sulfamoyl)amide, according to any one of embodiments 1) to 7), to be administered notably corresponds to 10 to 50 mg, especially 10 to 25 mg, in particular 12.5 mg or 25 mg, per day of aprocitentan in free acid form (wherein preferably said amount is (to be) administered once a day in the morning).

Thus, any pharmaceutical composition mentioned herein notably comprises sodium (5-(4-bromophenyl)-6-(2-((5-bromopyrimidin-2-yl)oxy)ethoxy)pyrimidin-4-yl)(sulfamoyl)amide according to any one of embodiments 1) to 7) in a pharmaceutical unit dosage form suitable for the oral administration of an amount corresponding to 10 to 50 mg (especially 10 to 25 mg, in particular 12.5 mg or 25 mg) per day of aprocitentan (in free acid form).

Particular embodiments of the invention are described in the following Examples, which serve to illustrate the invention in more detail without limiting its scope in any way.

### EXPERIMENTAL PART

The following abbreviations are used throughout the specification and the examples:
° degree(s)
° C. degree(s) Celsius
EtOAc ethyl acetate
eq. equivalent(s)
g gram(s)
GVS gravimetric vapor sorption
h hour(s)
HPLC high-performance liquid chromatography
kg kilogram(s)
kV kilovolt(s)
L (or l) liter(s)
m/m mass-per-mass ratio
mA milliampere(s)
mbar millibar(s)
MeCN acetonitrile
MeOH methanol
mg milligram
min minute(s)
mL (or ml) millilitre(s)
mol mole(s)
mm millimeter(s)
NaOH sodium hydroxide
rpm revolutions per minute
s second(s)
RH (or r.h.) relative humidity
THF tetrahydrofuran
vol. volume(s)
w/w weight-per-weight ratio
wt. weight unit

All temperatures are stated in ° C.

### X-Ray Powder Diffraction (XRPD) Analysis

All XRPD patterns for the solid forms described herein have been obtained as described hereafter. X-ray diffractograms were measured on a Bruker D8 Advance diffractometer with FlipStick™ sample stage, Cu Kc radiation (40 kV, 40 mA), and 1D-linear LynxEye™ detector. Samples were prepared on a silicon single crystal sample holder with a cavity of 25 mm diameter and no, 0.2-, or 0.5-mm depth. The powder was spread with a microscope slide to obtain a flat surface. Diffractograms were collected in the reflection mode with coupled θ/2θ angles in the range from 3-50° 2θ, an increment of 0.02° and an accumulation time of 0.4 s per step. The divergence and the antiscatter slit were set to 0.3°. The samples were continuously rotated with 30 rpm during the measurement. 2θ values of peak positions are given with an accuracy of +/−0.2°. as it is generally the case for conventionally recorded X-ray powder diffraction patterns.

### Differential Scanning Calorimetry (DSC)

DSC data were collected on a Mettler Toledo STARe System (DSC822e module, measuring cell with ceramic sensor and STAR software version 9.20) equipped with a 34-position auto-sampler. The instrument was calibrated for energy and temperature using certified indium. Typically, 1-5 mg of each sample, in an automatically pierced aluminium pan, was heated at 10° C. min-1, unless stated otherwise, from −20° C. to 280° C. A nitrogen purge at 20 ml min-1 was maintained over the sample. Peak temperatures are reported for melting points.

### Thermogravimetric Analysis (TGA)

TGA data were collected on a Mettler Toledo STARe System (TGA851e module and STAR software version 9.20) equipped with a 34-position auto-sampler. Typically, about 5 mg of a sample, in an automatically pierced aluminium pan, was heated at 10° C. min⁻¹, unless stated otherwise, from 30° C. to 250° C. A nitrogen purge at 10 ml min⁻¹ was maintained over the sample.

### Gravimetric Vapor Sorption (GVS)

Hygroscopicity was characterized by measurement of the moisture sorption isotherm at 25° C. on a Hiden Analytical Ltd. IGASORP Model HAS-036-080. After equilibration at 40% r.h. the relative humidity over the sample was increased to 80% r.h. in 5% steps allowing for a minimal equilibration time of 720 min and a maximum of 1440 min per step. The hygroscopicity was based on the definitions as provided by the EUROPEAN PHARMACOPOEIA 7.0 “5.11. Characters section in monographs” with Not hygroscopic: increase in mass is less than 0.2 percent m/m; Slightly hygroscopic: increase in mass is less than 2 percent m/m and equal to or greater than 0.2 percent m/m; Hygroscopic: increase in mass is less than 15 percent m/m and equal to or greater than 2 percent m/m.

### Assay Determination by HPLC Analysis

The assay was determined by use of UV-HPLC (e.g., Shimadzu Nexera X2 System) with an autosampler temperature set at 25° C. and a column temperature set at 50° C. Typically 1 μL of sample is injected and separation is obtained by use of a Phenomenex Gemini 5.0 μm 110 Å, 50×2.0 mm column using a flow of 1.5 mL/min. Samples are typically prepared by solubilization in acetonitrile/carbonate buffer pH10 1/1. A gradient method is used with solvent A consisting of Trizma base buffer 50 mM pH 9 (adjusted with HCl) and solvent B consisting of Acetonitrile+5% (v/v) Trizma base buffer 50 mM pH 9 (adjusted with HCl). Following gradient is typically used:

| Time (min) | % solvent A | % solvent B |
|---|---|---|
| 0.000 | 98.0 | 2.0 |
| 2.000 | 5.0 | 95.0 |
| 2.200 | 5.0 | 95.0 |
| 2.210 | 98.0 | 2.0 |
| 2.500 | 98.0 | 2.0 |

### EXAMPLES

### Example 1a: COMPOUND in Crystalline Form 1

A mixture consisting of 5 mL MeOH, 50 mg of aprocitentan as free acid form and 1 eq. of 1 M NaOH mixture is evaporated. To the remaining oily residue 0.5 mL of MeOH and 0.5 mL of MeCN is added and the mixture is allowed to stand overnight, closed at ambient temperature. The solid residue is collected, washed with 0.2 mL MeCN, and dried for 1 h at reduced pressure of 2 mbar. The solid is COMPOUND in crystalline form 1.

### Example 1b: COMPOUND in Crystalline Form 1

250 mg of aprocitentan as free acid form is dissolved in 15 mL of THE and 5 mL of MeOH. To this solution 0.458 mL 1 M NaOH solution is added. The solution is allowed to stand for 5 min and is then concentrated on the rotavapor. The remaining oil is briefly dried under high vacuum, dissolved in 2 mL hot MeOH and 15 mL MeCN is added, and the mixture is allowed to stand closed at ambient conditions. After 3 hours the solid material is collected, washed with 2×2 mL MeCN and dried 1 h at 2 mbar. The solid is COMPOUND in crystalline form 1.

### Example 1c: COMPOUND in Crystalline Form 1

A reactor is charged with aprocitentan as free acid form (3.5 kg, 6.4 mol, 1.0 eq.) and MeOH (24.9 kg, 9.0 vol). Methanolic sodium methoxide (30%; 1.16 kg, 6.4 mol, 1.0 eq.) is added, rinsing with MeOH (2.8 kg, 1.0 vol). The mixture is agitated at 20° C. for 4 h, then concentrated to a residual volume of 14 L (4 vol.) at 20° C. and 100 mbar. EtOAc (31.5 kg, 10.0 vol.) is added and more solvent is removed by distillation under the same conditions to a final volume of 21 L (6 vol.). EtOAc (15.8 kg, 5 vol.) is added, the mixture is agitated at 20° C. overnight, then at 0° C. for 2 h. Filtration, washing with cold EtOAc (20 kg, 5.7 vol.) and drying at 30-55° C. affords COMPOUND (3.15 kg, 87% yield) as a white solid material. The solid is COMPOUND in crystalline form 1. Data are summarized in the Table below.

TABLE 1
Characterization data for COMPOUND in crystalline form 1

| Technique | Data Summary | Remarks |
|---|---|---|
| XRPD | Crystalline | see FIG. 1 |
| 1H-NMR | Consistent | |
| DSC | A small endothermal event in the range of | |
| | about 109° C. to 138° C. is followed | |
| | by a large exothermal event | |
| | in the range from 178° C. to 220° C. | |
| TGA | Mass loss of 0.5% in the range 35° C. to | |
| | 225° C. | |
| GVS | Hygroscopicity classification: Slightly | |
| | hygroscopic | |

### Example 2: COMPOUND in Crystalline Form 2

COMPOUND in crystalline form 1 is stored at 23° C. and 92% r.h. for 9 weeks. The solid is immediately measured by XRPD. Data are summarized in the Table below. The solid is COMPOUND in crystalline form 2.

TABLE 2
Characterization data for COMPOUND in crystalline form 2

| Technique | Data Summary | Remarks |
|---|---|---|
| XRPD | Crystalline | see FIG. 2 |
| TGA | Mass loss of 0.9% in the range 34 to 199° C. | |

### Example 3: COMPOUND in Crystalline Form 3

A solution consisting of 9.2 mL 0.1 M NaOH, 500 mg of aprocitentan free acid form, and 25 mL MeCN is evaporated to dryness at 40° C. on a rotavapor. 5 mL of MeOH is added to the solid residue and stirred at ambient temperature. After 2-3 h the suspension is filtered and the immediately analyzed solid is COMPOUND in crystalline form 3.

TABLE 3
Characterization data for COMPOUND in crystalline form 3

| Technique | Data Summary | Remarks |
|---|---|---|
| XRPD | Crystalline | see FIG. 3 |

### Example 4: COMPOUND in Crystalline Form 4

A reactor is charged with aprocitentan free acid form (3.5 kg, 6.4 mol, 1.0 eq.) and MeOH (24.9 kg, 9.0 vol). Methanolic sodium methoxide (30%; 1.16 kg, 6.4 mol, 1.0 eq.) is added, rinsing with MeOH (2.8 kg, 1.0 vol). The mixture is agitated at 20° C. for 4 h, then transferred out to a rotary evaporator and stripped at 30° C. The stripped product is returned to the reactor, rinsing in with ethyl acetate (31.5 kg, 10 vol.). The mixture is agitated at 20° C. for 2 hours, cooled to 0-5° C. and the product is filtered off, washing with cold ethyl acetate (7 L, 2.0. vol.), furnishing wet product which is then dried under vacuum at 30° C. for 4 days with nitrogen bleed giving a dry weight of 3.05 kg (84% yield). The final solid material is COMPOUND in crystalline form 4.

TABLE 4
Characterization data for COMPOUND in crystalline form 4

| Technique | Data Summary | Remarks |
|---|---|---|
| XRPD | Crystalline | see FIG. 4 |

### Comparative Stability Experiments

Aprocitentan in free form (aprocitentan in crystalline Form A) (prepared according to Example 1 of WO2018/154101) and COMPOUND in crystalline form 1 according to the present invention were subjected to head-to-head comparative stability assessment comprising storing samples of the above-mentioned materials in parallel in open vials at certain temperatures and relative humidities (RH), and determining assay in the stored samples by HPLC (method describe hereinabove) at different time points. The results are summarized in the Table below.

TABLE 5

| | | Aprocitentan in free form | | COMPOUND in | |
|---|---|---|---|---|---|
| | | (in crystalline Form A) | | crystalline form 1 | |
| Initial assay [%] | | 96.9 | | 104.1 | |
| Test point | | 11% RH | 75% RH | 11% RH | 75% RH |
| 40° C. | 7 days | 97.2 | 91.8 | 104.4 | 104.0 |
| 60° C. | 7 days | 96.7 | 93.7 | 104.5 | 102.6 |
| 80° C. | 3 days | 89.7 | 0.3 | 103.4 | 72.4 |

The data presented in Table 5 show that under certain experimental conditions the COMPOUND in crystalline form 1 has a superior chemical stability in comparison to aprocitentan free acid form (in crystalline Form A).

1. A crystalline form of the compound sodium (5-(4-bromophenyl)-6-(2-((5-bromopyrimidin-2-yl)oxy)ethoxy)pyrimidin-4-yl)(sulfamoyl)amide 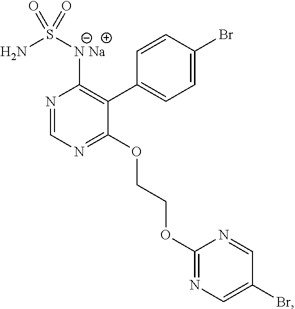
wherein
form 1 of the compound is characterized by the presence of at least four, or at least six, or at least eight peaks in the X-ray powder diffractogram at angles of refraction 2θ selected from: 4.3°, 13.1°, 13.5°, 14.8°, 17.2°, 19.5°, 21.1°, 21.6°, 22.6°, and 25.0°;
form 2 of the compound is characterized by the presence of at least four, or at least six, or at least eight peaks in the X-ray powder diffractogram at angles of refraction 2θ selected from: 4.2°, 13.3°, 16.7°, 16.9°, 18.9°, 20.8°, 22.5°, 23.5°, 25.6°, and 27.2°; or
form 3 of the compound is characterized by the presence of at least four, or at least six, or at least eight peaks in the X-ray powder diffractogram at angles of refraction 2θ selected from: 3.9°, 11.9°, 15.0°, 15.8°, 17.0°, 19.8°, 22.4°, 23.3°, 23.8°, and 27.8°;
wherein said X-ray powder diffractogram is obtained by using combined Cu Kα1 and Kα2 radiation, without Kα2 stripping; and the accuracy of the 2θ values is in the range of +/−0.1-0.2°.

2. A crystalline form of the compound sodium (5-(4-bromophenyl)-6-(2-((5-bromopyrimidin-2-yl)oxy)ethoxy)pyrimidin-4-yl)(sulfamoyl)amide, wherein
form 1 of the compound is characterized by the presence of peaks in the X-ray powder diffractogram at the following angles of refraction 2θ: 4.3°, 17.2°, and 22.6°;
form 2 of the compound is characterized by the presence of peaks in the X-ray powder diffractogram at the following angles of refraction 2θ: 13.3°, 20.8°, and 22.5; or
form 3 of the compound is characterized by the presence of peaks in the X-ray powder diffractogram at the following angles of refraction 2θ: 3.9°, 15.8°, and 19.8°;
wherein said X-ray powder diffractogram is obtained by using combined Cu Kα1 and Kα2 radiation, without Kα2 stripping; and the accuracy of the 2θ values is in the range of +/−0.1-0.2°.

3. A crystalline form of the compound sodium (5-(4-bromophenyl)-6-(2-((5-bromopyrimidin-2-yl)oxy)ethoxy)pyrimidin-4-yl)(sulfamoyl)amide according to claim 2, wherein
form 1 of the compound is characterized by the presence of peaks in the X-ray powder diffractogram at the following angles of refraction 2θ: 4.3°, 17.2°, 21.1°, 21.6°, and 22.6°;
form 2 of the compound is characterized by the presence of peaks in the X-ray powder diffractogram at the following angles of refraction 2θ: 4.2°, 13.3°, 18.9°, 20.8°, and 22.5°; or
form 3 of the compound is characterized by the presence of peaks in the X-ray powder diffractogram at the following angles of refraction 2θ: 3.9°, 15.8°, 19.8°, 23.8°, and 27.8°;
wherein said X-ray powder diffractogram is obtained by using combined Cu Kα1 and Kα2 radiation, without Kα2 stripping; and the accuracy of the 2θ values is in the range of +/−0.1-0.2°.

4. A crystalline form of the compound sodium (5-(4-bromophenyl)-6-(2-((5-bromopyrimidin-2-yl)oxy)ethoxy)pyrimidin-4-yl)(sulfamoyl)amide according to claim 3, wherein
form 1 of the compound is characterized by the presence of peaks in the X-ray powder diffractogram at the following angles of refraction 2θ: 4.3°, 13.1°, 13.5°, 14.8°, 17.2°, 19.5°, 21.1°, 21.6°, 22.6°, and 25.0°;
form 2 of the compound is characterized by the presence of peaks in the X-ray powder diffractogram at the following angles of refraction 2θ: 4.2°, 13.3°, 16.7°, 16.9°, 18.9°, 20.8°, 22.5°, 23.5°, 25.6°, and 27.2°; or
form 3 of the compound is characterized by the presence of peaks in the X-ray powder diffractogram at the following angles of refraction 2θ: 3.9°, 11.9°, 15.0°, 15.8°, 17.0°, 19.8°, 22.4°, 23.3°, 23.8°, and 27.8°;
wherein said X-ray powder diffractogram is obtained by using combined Cu Kα1 and Kα2 radiation, without Kα2 stripping; and the accuracy of the 2θ values is in the range of +/−0.1-0.2°.

5. A crystalline form of the compound sodium (5-(4-bromophenyl)-6-(2-((5-bromopyrimidin-2-yl)oxy)ethoxy)pyrimidin-4-yl)(sulfamoyl)amide, which essentially shows the X-ray powder diffractogram of:
form 1 depicted in FIG. 1;
form 2 depicted in FIG. 2; or
form 3 depicted in FIG. 3;
wherein said X-ray powder diffractogram is obtained by using combined Cu Kα1 and Kα2 radiation, without Kα2 stripping; and the accuracy of the 2θ values is in the range of +/−0.1-0.2°.

6. (canceled)

7. A pharmaceutical composition comprising as active ingredient a crystalline form of the compound sodium (5-(4-bromophenyl)-6-(2-((5-bromopyrimidin-2-yl)oxy)ethoxy)pyrimidin-4-yl)(sulfamoyl)amide, according to claim 1, and at least one therapeutically inert excipient.

8. A method for the treatment of hypertension, pulmonary hypertension, a coronary disease, cardiac insufficiency, renal ischemia, myocardial ischemia, renal failure, cerebral ischemia, dementia, migraine, subarachnoidal hemorrhage, Raynaud's syndrome, digital ulcers, or portal hypertension, in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

9. (canceled)

10. A method for the treatment of essential hypertension, resistant hypertension, pulmonary hypertension, or pulmonary arterial hypertension, in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

11. A method for the treatment of resistant hypertension in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

12. (canceled)

13. A method for the treatment of hypertension; CKD; acute or chronic renal failure; diabetic nephropathy; glomerulonephritis; or heart failure (HF); in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

14. A method for the treatment of resistant hypertension in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

15. A method for the treatment or prevention of atherosclerosis, restenosis after balloon or stent angioplasty, inflammation, stomach ulcer, duodenal ulcer, cancer, melanoma, prostate cancer, prostatic hypertrophy, erectile dysfunction, hearing loss, amaurosis, chronic bronchitis, asthma, pulmonary fibrosis, gram negative septicemia, shock, sickle cell anemia, glomerulonephritis, renal colic, glaucoma, a connective tissue disease, a diabetic complication, a complication of vascular or cardiac surgery or after organ transplantation, a complication of cyclosporin treatment, pain, or hyperlipidemia, in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

16. A method for the treatment of difficult to treat/resistant hypertension; CKD caused by/associated with hypertension; CKD caused by/associated with diabetes (DKD); or chronic heart failure; in a patient in need thereof, wherein the method comprises administering to the patient an effective amount of a crystalline form of the compound sodium (5-(4-bromophenyl)-6-(2-((5-bromopyrimidin-2-yl)oxy)ethoxy)pyrimidin-4-yl)(sulfamoyl)amide, according to claim 1.

17. A process for preparing a pharmaceutical composition comprising as active ingredient {5-(4-bromo-phenyl)-6-[2-(5-bromo-pyrimidin-2-yloxy)-ethoxy]-pyrimidin-4-yl}-sulfamide, wherein the process comprises admixing a crystalline form of the compound sodium (5-(4-bromophenyl)-6-(2-((5-bromopyrimidin-2-yl)oxy)ethoxy)pyrimidin-4-yl)(sulfamoyl)amide according to claim 1 and at least one therapeutically inert excipient.
